(19) United States
(12) Patent Application Publication
(10) Pub. No.: US 2021/0205384 A1
(43) Pub. Date: Jul. 8, 2021

(54) ECHO-7 ONCOLYTIC VIRUS FOR USE IN THE TREARMENT OF UVEAL MELANOMA

(71) Applicant: DITESAN LTD., Riga (LV)

(72) Inventors: Dite Venskus, Jelgava (LV); Ivars Kalvins, Ikskile (LV); Peteris Alberts, Riga (LV); Karlis Urbans, Garciems (LV); Jurgis Auzins, Olaine (LV)

(73) Assignee: DITESAN LTD., Riga (LV)

(21) Appl. No.: 17/057,230

(22) PCT Filed: May 23, 2019
(86) PCT No.: PCT/EP2019/063360

§ 371 Date: Nov. 20, 2020

(30) Foreign Application Priority Data
May 23, 2018 (GB) 1808500.1

(51) Int. Cl. A61K 35/768; A61K 9/00; A61P 35/00; C12N 7/00

(52) U.S. Cl. CPC A61K 35/768; A61K 9/0019; A61K 9/0014; C12N 2770/32332; A61P 35/00; C12N 7/00; A61K 9/0048

(57) ABSTRACT

Provided herein is the use of an ECHO-7 class oncolytic virus for the treatment of uveal melanoma and compositions comprising the ECHO-7 virus and pharmaceutically acceptable carrier for the treatment of uveal melanoma.

### TECHNICAL FIELD

The present invention relates to methods and compositions for the treatment of uveal melanoma by administering compositions comprising an ECHO-7 class oncolytic virus and a pharmaceutically acceptable carrier.

### STATE OF THE ART

Treatment of uveal melanoma is an unsolved problem in oncology because uveal melanoma often develops metastases and is incurable (Singh A D, Turell M E, Topham A K. Uveal melanoma: trends in incidence, treatment, and survival. Ophthalmology 2011; 118:1881-5). Despite some efforts in treatment of cutaneous melanoma, attempts to use medicines approved for cutaneous melanoma to treat uveal melanoma were with very disappointing results (Pereira P R, Odashiro A N, Lim L A, et al. Current and emerging treatment options for uveal melanoma. Clin Ophthalmol 2013; 7:1669-82). This is understandable, because cutaneous and uveal melanomas are biologically substantially distinct (for review see: Carvajal R D, et al. Br J Ophthalmol 2017; 101:38-44. doi:10.1136/bjophthalmo1-2016-309034).

Despite the development of some local therapies, 5-year survival rates (˜80%) of uveal melanoma have not changed in the past three decades (Singh A D, Turell M E, Topham A K. Uveal melanoma: trends in incidence, treatment, and survival. Ophthalmology 2011; 118:1881-5) and up to 50% of patients develop metastases (Kujala E, Makitie T, Kivelä T. Very long-term prognosis of patients with malignant uveal melanoma. Invest Opthalmol Vis Sci 2003; 44:4651).

No effective adjuvant systemic therapy has been demonstrated to reduce the risk of metastasis (Triozzi P L, Singh A D. Adjuvant therapy of uveal melanoma: current status. Ocul Oncol Pathol 2015; 1:54-5.) One-year survival of patients with metastases is reported to be 15%, with reported median survival ranging from 4 to 15 months (Collaborative Ocular Melanoma Study Group. The COMS randomized trial of iodine 125 brachytherapy for choroidal melanoma: V. Twelve-year mortality rates and prognostic factors: COMS report No. 28. Arch Ophthalmol 2006; 124:1684-93).

For those patients who do develop metastatic disease, there is yet no proven standard of care (Carvajal R D, Schwartz G K, Tezel T, et al. Metastatic disease from uveal melanoma: treatment options and future prospects. Br J Ophthalmol 2017; 101:38-44).

Dacarbazine, a chemotherapeutic option for treatment of cutaneous melanoma, has been used for uveal melanoma, despite the inherent differences between these distinct diseases (Nathan P, Cohen V, Coupland S, et al. Uveal Melanoma National Guidelines. 2015. http://melanomafocus.com/activities-2/um-guidelines-resources/. Dummer, R., Hauschild, A., Lindenblatt, N., Pentheroudakis, G., Keilholz, U., 2015. Cutaneous melanoma: ESMO Clinical Practice Guidelines for diagnosis, treatment and follow-up. Ann. Oncol. 26 Suppl 5, v126-v132. https://doi.org/10.1093/annonc/mdv297. 42 NCCN. NCCN, 2018. National Comprehensive Cancer Network. Clinical practice guidelines in oncology. Melanoma. V2. February 21 (https://www.nccn.org/professionals/physician_gls/pdf/melanoma_blocks.pdf). 1-173.).

As to be expected, the activity of dacarbazine in treatment of uveal melanoma is limited (Dummer, R., Hauschild, A., Lindenblatt, N., Pentheroudakis, G., Keilholz, U., 2015. Cutaneous melanoma: ESMO Clinical Practice Guidelines for diagnosis, treatment and follow-up. Ann. Oncol. 26 Suppl 5, v126-v132. https://doi.org/10.1093/annonc/mdv297. Carvajal R D, Sosman J A, Quevedo J F, et al. Effect of selumetinib vs chemotherapy on progression-free survival in uveal melanoma: a randomized clinical trial. JAMA 2014; 311:2397-405. Sacco J J, Nathan P D, Danson S, et al. Sunitinib versus dacarbazine as first-line treatment in patients with metastatic uveal melanoma. J Clin Oncol 2013; 31 (Suppl): Abstr 9031).

Other chemotherapeutic regimens including temozolomide, cisplatin, treosulfan, fotemustine and combinations thereof have been investigated in uveal melanoma with disappointing results to date (Augsburger J J, Corrêa Z M, Shaikh A H. Effectiveness of treatments for metastatic uveal melanoma. Am J Ophthalmol 2009; 148:119-27. Pereira P R, Odashiro A N, Lim L A, et al. Current and emerging treatment options for uveal melanoma. Clin Ophthalmol 2013; 7:1669-82. Spagnolo F, Grosso M, Picasso V, et al. Treatment of metastatic uveal melanoma with intravenous fotemustine. Melanoma Res 2013; 23:196-8).

Ipilimumab, a human monoclonal antibody that blocks the cytotoxic T-lympho-cyte-associated antigen 4 (CTLA-4), is approved in the USA and Europe for the treatment of advanced, unresectable melanoma (Dummer, R., Hauschild, A., Lindenblatt, N., Pentheroudakis, G., Keilholz, U., 2015. Cutaneous melanoma: ESMO Clinical Practice Guidelines for diagnosis, treatment and follow-up. Ann. Oncol. 26 Suppl 5, v126-v132. https://doi.org/10.1093/annonc/mdv297).

Response rates of ˜5%-10% have been reported from evaluations in metastatic uveal melanoma (Deo M A. Long-term survival benefit from ipilimumab treatment in metastatic uveal melanoma patients. J Clin Oncol 2014; 32 (Suppl): Abstract 3060. Khan S A, Callahan M, Postow M A, et al. Ipilimumab in the treatment of uveal melanoma: the Memorial Sloan-Kettering Cancer Center experience. J Clin Oncol 2012; 30 (Suppl): Abstract 8549. Luke J J, Callahan M K, Postow M A, et al. Clinical activity of ipilimumab for metastatic uveal melanoma: a retrospective review of the Dana-Farber Cancer Institute, Massachusetts General Hospital, Memorial Sloan-Kettering Cancer Center, and University Hospital of Lausanne experience. Cancer 2013; 119:3687-95. Maio M, Danieffi R, Chiarion-Sileni V, et al. Efficacy and safety of ipilimumab in patients with pre-treated, uveal melanoma. Ann Oncol 2013; 24:2911-15).

Once more the results of these trials confirmed that cutaneous and uveal melanoma do not react similarly to same therapy. Thus, evidence of a median overall survival (OS) of 6.0-9.7 months in these trials suggests that responses could be delayed and durable in only a minority of patients (Khan S A, Callahan M, Postow M A, et al. Ipilimumab in the treatment of uveal melanoma: the Memorial Sloan-Kettering Cancer Center experience. J Clin Oncol 2012; 30 (Suppl): Abstract 8549. Luke J J, Callahan M K, Postow M A, et al. Clinical activity of ipilimumab for metastatic uveal melanoma: a retrospective review of the Dana-Farber Cancer Institute, Massachusetts General Hospital, Memorial Sloan-Kettering Cancer Center, and University Hospital of Lausanne experience. Cancer 2013; 119:3687-95.)

According to results of clinical trials, ipilimumab demonstrated very limited clinical activity in treatment-naïve or pre-treated patients with metastatic uveal melanoma-median progression-free survival (PFS) was 2.8 months and median OS was 6.8 months (Zimmer L, Vaubel J, Mohr P, et al. Phase II DeCOG-study of ipilimumab in pretreated and treatment-naive patients with metastatic uveal melanoma. PLoS ONE 2015; 10:e0118564).

Attempts were made to use another class of biological medicines for treatment of cutaneous melanoma—nivolumab and pembrolizumab. These drugs, fully human monoclonal antibodies targeting the programmed cell death 1 (PD-1) receptor are approved in the USA and Europe for advanced cutaneous melanoma. However, the activity of PD-1 inhibition in uveal melanoma did not substantially improve the results of treatment. (Robert C, Ribas A, Wolchok J D, et al. Anti-programmed-death-receptor-1 treatment with pembrolizumab in ipilimumab-refractory advanced melanoma: a randomised dose-comparison cohort of a phase 1 trial. Lancet 2014; 384:1109-17; Robert C, Long G V, Brady B, et al. Nivolumab in previously untreated melanoma without BRAF mutation. N Engl J Med 2015; 372:320-30. 55 Weber J S, D'Angelo S P, Minor D, et al. Nivolumab versus chemotherapy in patients with advanced melanoma who progressed after anti-CTLA-4 treatment (CheckMate 037): a randomised, controlled, open-label, phase 3 trial. Lancet Oncol 2015; 16:375-84).

Recently some novel options for uveal melanoma treatment were disclosed. Application of 2-methoxyestradiol to preparation of medicine for treating uveal malignant melanoma CN107375301 (A)—2017 Nov. 24; Application of CTCF snare protein in preparation of anti-uveal melanoma medicine CN106913861 (A)—2017 Jul. 4; Application of pan-HDAC inhibitor to preparation of drug used for treating uveal melanoma CN106902106 (A)—2017 Jun. 30; Method For The Treatment Of Proliferative Disorders Of The Eye US 2011200662 (A1)/. Nevertheless, these approaches are not approved by any regulatory body for clinical use in humans.

Both single-stranded and double-stranded RNA and DNA viruses are recently recognized as promising oncolytic agents for cancer treatment/Fountzilas Ch., Patel S., Mahalingam D. Review: Oncolytic virotherapy, updates and future directions. Oncotarget. 2017; 8:102617-102639. https://doi.org/10.18632/oncotarget.18309/.

Among single-stranded RNA viruses, Coxsackie virus A21 and B3 were tested as anti cancer agents binding to the DAF receptor/Harrington K J, Karapanagiotou E M, Roulstone V, Twigger K R, White C L, Vidal L, Beirne D, Prestwich R, Newbold K, Ahmed M, Thway K, Nutting C M, Coffey M, et al. Two-stage phase I dose-escalation study of intratumoral reovirus type 3 dearing and palliative radiotherapy in patients with advanced cancers. Clin Cancer Res. 2010; 16:3067-77. https://doi.org/10.1158/1078-0432.CCR-10-0054; Cheung N K, Walter E I, Smith-Mensah W H, Ratnoff W D, Tykocinski M L, Medof M E. Decay-accelerating factor protects human tumor cells from complement-mediated cytotoxicity in vitro. J Clin Invest. 1988; 81:1122-28. https://doi.org/10.1172/JCI113426; Li L, Spendlove I, Morgan J, Durrant L G. CD55 is over-expressed in the tumor environment. Br J Cancer. 2001; 84:80-86. https://doi.org/10.1054/bjoc.2000.1570; Shafren D R, Dorahy D J, Ingham R A, Burns G F, Barry R D. Coxsackievirus A21 binds to decay-accelerating factor but requires intercellular adhesion molecule 1 for cell entry. J Virol. 1997; 71:4736-43/.

There is some evidence that Coxsackie virus A21 induces oncolysis of vascular malignant human melanoma tumors/Au G G, Lindberg A M, Barry R D, Shafren D R. Oncolysis of vascular malignant human melanoma tumors by Coxsackievirus A21. Int J Oncol. 2005; 26:1471-76; Andtbacka R H, Curti B D, Kaufman H, Daniels G A, Nemunaitis J J, Spitler L E, Hallmeyer S, Lutzky J, Schultz S M, Whitman E D, Zhou K, Karpathy R, Weisberg J I, et al. Final data from CALM: A phase II study of Coxsackievirus A21 (CVA21) oncolytic virus immunotherapy in patients with advanced melanoma. ASCO Meeting Abstracts. 2015; 33: 9030/

Other oncolytic RNA, single-strand viruses (Vesicular Stomatitis virus, Seneca Valley virus) have not been tested on melanoma cancer up-to-day. None of the viruses has been tested on uveal melanoma.

Echoviruses are single-stranded RNA viruses and demonstrated anticancer activity on human ovarian cancers as well as on human prostate cancer/Shaken D R, Sylvester D, Johansson E S, Campbell I G, Barry R D. Oncolysis of human ovarian cancers by echovirus type 1. Int J Cancer. 2005; 115:320-28. https://doi.org/10.1002/ijc.20866; Berry L J, Au G G, Barry R D, Shafren D R. Potent oncolytic activity of human enteroviruses against human prostate cancer. Prostate. 2008; 68:577-87. https://doi.org/10.1002/pros.20741/.

ECHO-7 virus is known to possess oncolytic activity in cutaneous melanoma. See for example document EP 1537872, and document WO 2015/007788 together with its corresponding patent EP 2826856. A non-pathogenic virus Picornaviridae family, Enterovirus genus, Enteric Cytopathogenic Human Orphan group, type 7 (ECHO-7), group VII, positive-sense single-stranded RNA virus adapted to melanoma, with oncolytic, oncotropic and immunomodulating properties is described in /Doni [symbol] a S, Strele I, Proboka G, et al. Melanoma Res. 2015; 25(5):421-426. Brūivere R, Heisele O, Ferdats A, Rupais A, Muceniece A. Acta Medica Lituanica Suppl. 2002; 9:97-100. Alberts P, Tilgase A, Rasa A, Bandere K, Venskus D. Eur J Pharmacol 2018; 837:117-126. Glinkina L S, Bruvere R Z, Venskus D R, Garklava R R, Muceniece A J. Vopr Onkol. 1992; 38(5): 540-547. Glinkina L S, Heisele O G, Garklava R R, Muceniece A J. Vopr Onkol. 1992; 38(5):534-540. Glinkina L S, Bruvere R Z. Vopr Onkol. 1992; 38 (6):659-666./.

ECHO-7 is active against a number of cancer cell lines in vitro, including human cutaneous melanoma cell line FM-9; ECACC 13012416; /Tilgase A., Patetko L., Blā [symbol] e I., Ramata-Stunda A., Borodš [symbol] is M., Alberts P, Journal of Cancer 2018; 9(6): 1033-1049. doi: 10.7150/jca.23242/.

There is some evidence that ECHO-7 may be active also against advanced cutaneous melanoma, small cell lung cancer and histiocytic sarcoma in humans. /Alberts P, Olmane E, Brokāne L, et al. APMIS. 2016; 124(10):896-904; Donina S, Strēle I, Proboka G, Auzi [symbol] š J, Alberts P, Jonson B, Venskus D, Muceniece A. Melanoma Res. 2015; 25:421-26. https://doi.org/10.1097/CM R.0000000000000180/.

Nevertheless, nothing is known about the activity of ECHO-7 or other oncolytic viruses in uveal melanoma, which is biologically distinct from cutaneous type of melanoma. Thus, no cure for cutaneous melanoma has so far been shown to be successful in the treatment of uveal melanoma.

Because of the limited activity of currently approved agents for advanced melanoma in the treatment of metastatic uveal melanoma there is a real need for specifically approved treatments and dedicated management strategies in order to improve outcomes for patients affected by this difficult-to-manage disease. Conventional anti-proliferative drugs such as adriamycin, cisplatin, taxol, vincristine, mitomycin, busulfan, Actinomycin-D, and phosphoramide mustards are highly cytotoxic, kill proliferating as well as nonproliferating cells, and cause tissue damage after local administration. In addition, such agents damage cells and can impair cell function. Earlier developed methods for local treatment of uveal melanoma can be applied only in early stage uveal melanoma without metastasis in liver or other organs and tissues. Therefore, effective, non-toxic and systemic active drugs are of high medical need.

### DESCRIPTION OF INVENTION

Unexpectedly we have found that the non-pathogenic oncolytic ECHO-7 type virus, which is the subject of EP 2826856 is effective in treatment of uveal melanoma. For the purpose of the present invention, ECHO-7 type virus means the modified enterovirus of the ECHO 7 type having a stable genome sequence as disclosed in EP 2826856 B1 and is obtainable according to the methods disclosed in EP 2826856 B1. The term “ECHO-7 type virus” as used herein in the context of instant invention mean the said non-pathogenic oncolytic ECHO-7 type virus as disclosed in EP 2826856 B1.

The term “TCID₅₀/ml” according to instant invention means the tissue culture infective dose 50 per millilitre with respect to MRC-5 cells (ATCC, CCL 171™) or RD cells (ATCC, CCL 136™), that is, the median tissue culture infective dose per millilitre.

### EXAMPLES

ECHO-7 type virus was obtained from SIA Latima in Riga, Latvia, as marketed under the trademark RIGVIR®.

“%”, if not otherwise indicated means % (v/v).

### Example 1. Cytolytic Effect of ECHO-7 on Uveal Melanoma Cell Viability

First, we found that ECHO-7 type virus very effectively kills uveal melanoma cells in vitro as described in Example 1, FIG. 1.) Experiments were performed according to previously described methods /Tilgase A., Patetko L., Blā [symbol] e I., Ramata-Stunda A., Boroduš [symbol] is M., Alberts P Journal of Cancer 2018; 9(6): 1033-1049.doi:10.7150/jca.23242/.

Cell Culture Conditions

Cell lines of human uveal melanoma were obtained as follows: 92-1 cells from Sigma Aldrich, Mel 202 from Sigma Aldrich, and MP41 from ATCC (American Type Culture Collection).

When the cell monolayer had reached approximately 10% confluency, ECHO-7 type virus (stock titre 10⁶-10⁷ TCID₅₀/ml) was added at final concentrations of 1% and 10% to the cell cultivation medium. Inactivated ECHO-7 type virus was used as a negative control. An equal volume of medium (without ECHO-7 type virus) was added to control cells; the cells were subsequently observed for 96 h.

Calculation of the Cytolytic Effect of ECHO-7 on Cell Viability.

The inhibitory effect of ECHO-7 type virus on viable cell count was calculated using the formula: 100−(100×A/B)=reduction (%), where A is cell count with ECHO-7 type virus, and B is the control cell count.

Statistics

The statistical difference between treatment groups was calculated using GraphPad Prism 7.02 software. Two-way ANOVA test was applied in the cell viability experiment.

Cytolytic Effect of ECHO-7 on Cell Viability

After incubation with ECHO-7 type virus (10%) of 92-1, Mel 202 and MP41 cell line cells almost no viable cells were observed by 96 h. The effect of ECHO-7 type virus (1%) was slower in onset than ECHO-7 type virus 10%.

### FIGURES

FIG. 1. Effect of ECHO-7 type virus on viable cell count 0 to 96 h. Statistical difference in cell viability between ECHO-7 type virus treated and control was observed from the following time-points.

Statistically significantly difference vs. control P<0.05 (*), not statistically different (n.s.). Data are expressed as means±S.E.M.

Control (●), ECHO-7 type virus (1%) (▪) and ECHO-7 type virus (10%) (▴).

FIG. 1 A. Effect on 92-1 cells: ECHO-7 type virus (1%) at 44 h (P=0.0459) and ECHO-7 type virus (10%) at 14 h (P=0.0036).

FIG. 1 B. Effect on Mel202 cells: ECHO-7 type virus (1%) at 23 h (P=0.0260) and ECHO-7 type virus (10%) at 11 h (P=0.0083).

FIG. 1 C. Effect on MP-41 cells: ECHO-7 type virus (1%) at 27 h (P=0.0362) and ECHO-7 type virus (10%) at 23 h (P=0.0241).

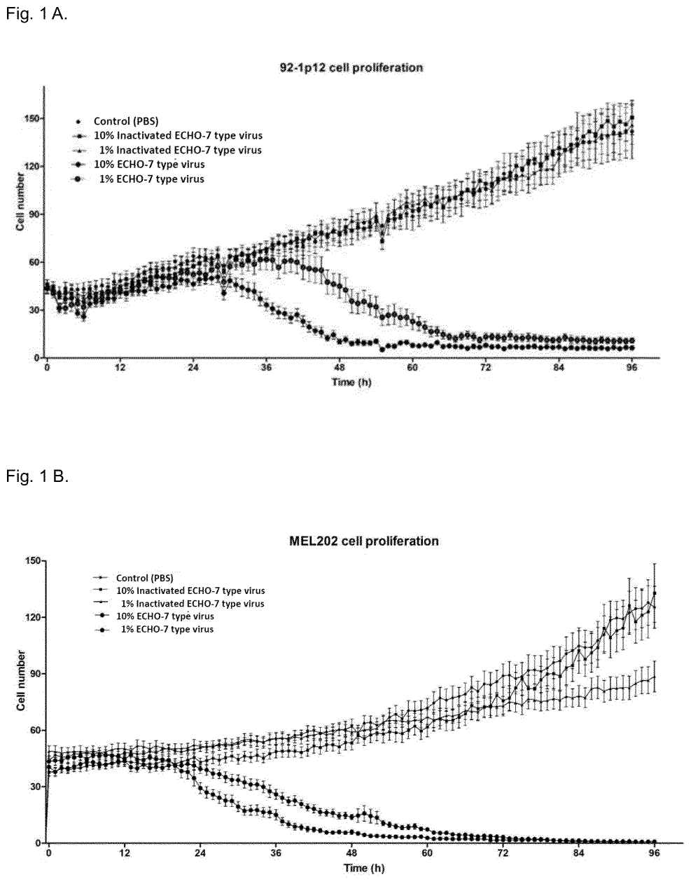

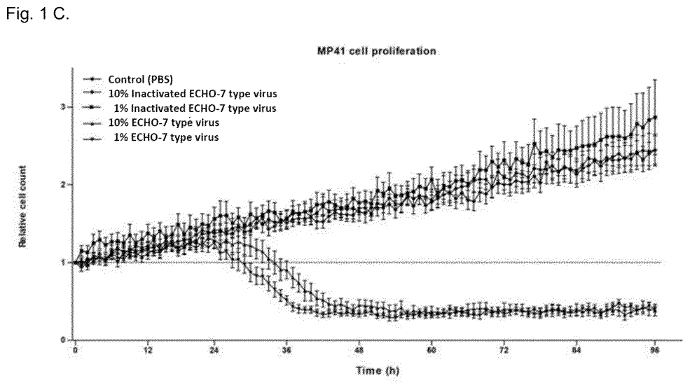

Treatment of Patients with Uveal Melanoma

The present invention includes methods for the treatment and prevention of uveal melanoma of the eye and metastases comprised of the following steps: selecting a pharmaceutical formulation comprising ECHO-7 type virus and an appropriate carrier; administration by injection or infusion or by applying topically of said drug directly into the eye or subcutaneously, intradermally, intramuscularly, intra-peritoneally, intranasally or intravenously at an effective amount, at an effective number of occasions, and for a sufficient period of time to invade cancer cells with ECHO-7.

The present invention includes further ECHO-7 type virus for use in the treatment and prevention of uveal melanoma of the eye and metastases thereof. As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, and the like and combinations thereof, as would be known to those skilled in the art (see, e.g., Remington's Pharmaceutical Sciences, 22^{nd} Ed. Mack Printing Company, 1990, pp. 1289-1329). Except insofar as any conventional carrier is incompatible with Echovirus, its use for therapy in pharmaceutical compositions is contemplated.

Subcutaneous, intramuscular, intranasal, intradermal, intraperitoneal or intravenous injections or locally administering directly into the eye of appropriate formulation to achieve the desired therapeutic objective is used to deliver a sufficient quantity or effective amount of ECHO-7 type virus to the target space. Preferably, according to instant invention the pharmaceutical composition comprising ECHO-7 type virus is administered parenteral, retro-bulbar or by intravitreal injection.

As used herein, “locally administering” refers to the direct administration of a drug to a site (for example to a target space of the eye), as opposed to drug delivery to that site by a systemic route or intravascular route or eye drops or gels. As used herein, “physical carrier” refers to carrier wherein the drug is absorbed on the surface of the physical carrier. Examples of pharmaceutically acceptable physical carriers include but are not limited to, ophthalmological grade sponges, gauze, cellulose sponges and gels. Biocompatible physical carriers that can be used in the eye are well known to one skilled in the art. /Rautio, J. et al., Nat Rev Drug Discov., 7(3): 255-70 (March 2008); Yasukawa, T. et al., Adv Drug Deliv Rev, 57(14): 2033-46 (Dec. 13, 2005); and Bourges, J. L. et al., Adv Drug Deliv Rev, 58(11):1182-202 (Nov. 15, 2006)/.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutical composition” is defined herein to refer to a mixture or solution, suspensions or emulsions containing ECHO-7 type virus to be administered to a warm-blooded animal, e.g., a mammal or human, in order to prevent or treat uveal melanoma affecting the warm-blooded animal inclusive human.

Methods of injection into the eye are well known to one skilled in the art and is done with a needle or catheter inserted into the vitreal space. Micro-infusion techniques, micro-needles and micro-catheters can also be used and are well known to one skilled in the art.

The ECHO-7 type virus can be formulated in a pharmaceutical composition comprising an effective amount of a drug, and a pharmaceutically acceptable carrier both for parenteral or locally administration.

The carriers are “pharmaceutically acceptable” in that they are not deleterious to the recipient thereof in an amount used in the medicament. Pharmaceutically acceptable carriers, adjuvants and vehicles that may be used in the methods of this invention include, but are not limited to buffer substances, such as phosphates, glycine, water, salts or electrolytes, such as protamine sulphate, disodium hydrogen phosphate, potassium hydrogen phosphate, sodium chloride, zinc salts, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The formulation can be a solution, suspension, emulsion, gel, polymeric paste. The drugs can be administered in combination with commonly employed pharmacological excipients, which include but are not limited to, saline, aqueous buffers, cyclodextrins, sodium hyaluronate, and stabilizers that are well known to one skilled in the art. ECHO-7 type virus can be dissolved in sterile saline or water or a buffered salt solution. In a preferred embodiment for intravitreal injections the virus is dissolved in an ophthalmological formulation of 1% sodium hyaluronate. In a preferred embodiment no organic solvent is employed in the formulation, rather the drug is formulated as a dry powder, to which is added an aqueous solution such as saline or ophthalmological grade of 1% sodium hyaluronate prior to administration.

For intramuscular or intravenous administration ECHO-7 type virus is formulated as a solution for injection or a dry powder, to which is added distilled water or an aqueous solution of saline.

The concentration, volume and total dose of the drug is dependent upon the clinical condition to be treated and the desired pharmacological effect. For intravitreal injections the injection volume will typically be in the range of 1 μl to 100 μl. In a preferred embodiment the injection volume is 50 μl for intravitreal injections. Techniques for the determination of clinical drug doses and concentrations are well known to one skilled in the art. The drug may be administered by one or more local injections or by catheters placed within the eye and connected to micro-infusion pumps. Suitable catheters and micro-infusion pumps, and injection techniques are well known to one skilled in the art.

For intramuscular or intravenous administration ECHO-7 type virus usually is administered at a dose of 2 ml of 10⁶ to 10⁸ TCID₅₀/ml.

The treatment may include but is not limited to injections of ECHO-7 for 3 consecutive days every 4 weeks for an initial 3-month period. Thereafter, the treatment has to be continued monthly during the first year, every 6 weeks during the first half of the second year, every 2 months during the second half of the second year and every 3 months during the third year. The treatment can be varied in accordance to clinical response and overall condition of the patient.

### Example 2. Preliminary Results of Treatment of Uveal Melanoma Patients by Intramuscular Administration of ECHO-7

In a retrospective study ECHO-7 type virus containing composition at a dose of 2 ml of 10⁶ to 10⁸ TCID₅₀/ml was applied intramuscularly to six patients (4 women and 2 men, age 31 to 61 year) with uveal melanoma after previous local irradiation therapy. Injections of ECHO-7 type virus were performed intramuscularly for three consecutive days every 4 weeks for an initial 3-month period. Thereafter, the treatment has been continued monthly during the first year, every 6 weeks during the first half of the second year, every 2 months during the second half of the second year and every 3 months during the third year.

During the treatment period all patients were investigated for primary melanoma progression and for development of metastasis. In four of six patients (67%), no sign of disease progression was noted. No side effect was reported for patients treated with ECHO-7 type virus. Two patients of six (33%) developed metastasis in the liver. For one of them disease became lethal during the second year after beginning ECHO-7 type virus treatment.

### Example 3

Another group of five patients with uveal melanoma (1 man and 4 women, age 49 to 61 years) previously irradiated locally, received ECHO-7 type virus for one year by intramuscular injection (following administration route and dosages as described above). During the following two years these patients received ECHO-7 type virus locally. During all period of the treatment, patients were investigated for primary melanoma progression and for development metastasis. Progression free survival up to end of 36-month period of treatment was noted for five out of six patients (83%). One of patients suffered from development of metastasis in liver and died after 29 months.

### Example 4. Long-Term ECHO-7 Treatment of a Uveal Melanoma Patient

Uveal melanoma was diagnosed in a 56-year old woman in 2007. After irradiation remission of uveal melanoma was noted. In 2010 progression of disease locally was noted. Locally irradiation of the eye was performed, but progression of malignant process was not stopped. Therefore, enucleation of the eye along with the optic nerve were performed in October 2016. After surgery administration of ECHO-7 type virus intramuscularly was started following the protocol, described for first group of patients in Example 1. After three years of treatment, progression of disease was not observed. Treatment did not cause any side effect.

In summary: The experimental evidence shows that ECHO-7 type virus is an effective and very safe drug for the treatment of uveal melanoma patients. ECHO-7 type virus is appropriate for the preparation of a pharmaceutical composition to be administered locally as well as parenterally and can be used for the prophylaxis and treatment of primary tumour and metastasis of uveal melanoma.

1. Use of ECHO-7 type virus comprising composition in the treatment of uveal melanoma, selected from primary and metastatic uveal melanoma.

2. The use according to claim 1, wherein the uveal melanoma is treated by parenteral administration of the said ECHO-7 type virus comprising composition.

3. The use according to claim 1, wherein the uveal melanoma is treated by intraocular administration of the said ECHO-7 type virus comprising composition.

4. The use according to claim 2, wherein the uveal melanoma is treated by retrobular administration of the said ECHO-7 type virus comprising composition.

5. The use according to claim 3, wherein said ECHO-7 type virus comprising composition is administered by intravitreal injection.

6. The use according to claim 2, wherein the ECHO-7 type virus comprising composition is administered topically.

7. The use according to claim 1, wherein the ECHO-7 type virus is in an amount in the range of 2×10⁶ to 2×10⁸ TCID₅₀/ml per single dose.

8. ECHO-7 type virus for the treatment of uveal melanoma, which is selected from primary and metastatic uveal melanoma.

9. A pharmaceutical composition for the treatment of uveal melanoma comprising the ECHO-7 type virus of claim 8.

10. A pharmaceutical composition according to claim 9, comprising an amount of ECHO-7 type virus is in the range of 2×10⁶ to 2×10⁸ TCID₅₀/ml per single dose.

11. A pharmaceutical composition according to claim 9 for parenteral administration.

12. A pharmaceutical composition according to claim 11 for intraocular administration.

13. A pharmaceutical composition according to claim 11 for retro-bulbar administration.

14. A pharmaceutical composition according to claim 11 for administration by intravitreal injection.

15. A pharmaceutical composition according to claim 9 for topical administration.

16. A pharmaceutical composition according to claim 10 for parenteral administration.

17. A pharmaceutical composition according to claim 16 for intraocular administration.

18. A pharmaceutical composition according to claim 16 for retro-bulbar administration.

19. A pharmaceutical composition according to claim 16 for administration by intravitreal injection.

20. A pharmaceutical composition according to claim 10 for topical administration.
